(19) United States
(12) Patent Application Publication
(10) Pub. No.: US 2025/0263356 A1
(43) Pub. Date: Aug. 21, 2025

(54) PROCESS OF PRODUCING TOLUENE

(71) Applicant: UOP LLC, Des Plaines, IL (US)

(72) Inventors: Niki Naik Shah, Gurugram (IN); Ian G. Horn, Streamwood, IL (US); Srinivasan Ramanujam, Bengaluru (IN); Nirlipt Mahapatra, Gurgaon (IN); Johan Erik Elm Svensson, Bracknell (GB); David Lafyatis, Schaumburg, IL (US)

(21) Appl. No.: 19/045,294

(22) Filed: Feb. 4, 2025

(30) Foreign Application Priority Data
Feb. 15, 2024 (IN) 202411010584

(51) Int. Cl. C07C 7/04; C07C 5/367; C07C 7/09

(52) U.S. Cl. CPC C07C 7/04; C07C 5/367; C07C 7/09

(57) ABSTRACT

A process of producing toluene is disclosed. The process comprises contacting a hydrocarbonaceous feed stream with a dehydrogenation catalyst to produce a dehydrogenated effluent stream. The dehydrogenated effluent stream is separated into a vapor stream comprising hydrogen and a liquid stream. The liquid stream is fractionated to provide a fractionator overhead stream comprising C7-hydrocarbons and a fractionator bottoms stream comprising toluene. The fractionator overhead stream is passed to a stabilizer column to provide an offgas stream.

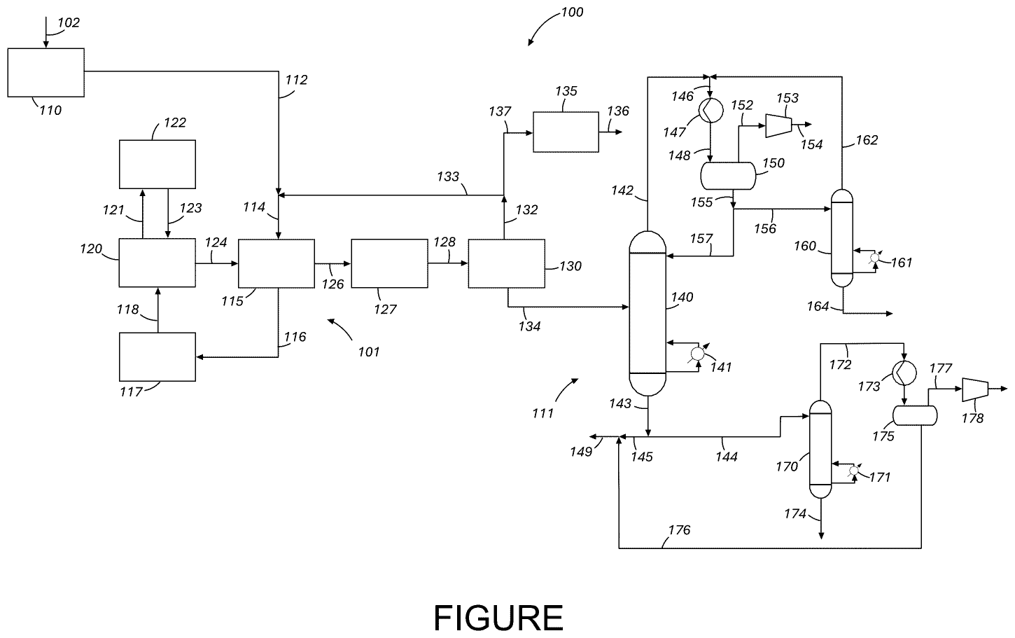

### FIELD

The field is the process of producing toluene. The field may particularly relate to separating hydrogen and toluene from a hydrocarbonaceous stream.

### BACKGROUND

Hydrogen is expected to have significant growth potential because it is a clean-burning fuel. However, hydrogen production processes based on steam reforming, autothermal reforming, partial oxidation, or gasification of hydrocarbon or carbonaceous feedstocks are significant emitters of carbon dioxide. Government regulations and societal pressures are increasingly taxing or penalizing carbon dioxide emissions or incentivizing carbon dioxide capture. Hydrogen produced by the electrolysis of water can be powered by solar and wind without involving the production of carbon dioxide. Hydrogen produced in this manner could meet projected global energy demand in the future and play a vital role in reducing carbon dioxide emissions. The recently renewed interest in alternative energy sources and energy carriers opens up new prospects for this process to be applied as a feed system for fuel cells, power generation and many more applications.

Hydrogen is a clean, efficient energy carrier in various mobile fuel-cell applications and has no adverse effects on the environment and human health. With the increase in global demand for hydrogen, solutions need to be developed to transport hydrogen especially to locations which are renewable depleted. Hydrogen generated by renewable energy is called green hydrogen. Green hydrogen is expected to be an important element in the future carbon-neutral economy and may need to be transported to locations as far as 8000 km from the source of generation.

There exists a large regional disparity in the cost of production of hydrogen. A number of technologies have been developed for transporting hydrogen, including ammonia, liquid hydrogen, and liquid organic hydrogen carrier (LOHC) to address this disparity. Toluene-methylcyclohexane (MCH) is expected to be a significant player in LOHC considering numerous advantages, such as easy integration into the existing fuel sector supply chain and distribution network, utilization in idle refinery assets, flexibility for co-processing, and higher relative safety handling.

LOHC involves the reversible dehydrogenation reaction of methylcyclohexane (MCH) to produce toluene (TOL) and hydrogen. It has been proposed as a solution for the storage, transportation, and distribution of hydrogen produced from renewable energy sources. For power generation, the hydrogen from this process is usually compressed for a downstream power generation unit. Typically, purity requirements for power generation unit are very stringent. Due to the relatively high cost associated with green hydrogen production, it is necessary to recover almost all hydrogen.

The process of recovering toluene from MCH is highly selective but may lead to the formation of byproducts that will build up in the carrier loop. These byproducts need to be removed before they cause problems with catalyst or equipment life. Also, the energy required to remove these byproducts must be minimized in order to avoid consuming valuable hydrogen product or consuming energy from carbon-emitting sources.

Accordingly, there is a need to have more effective and efficient ways to purify and transport hydrogen and in particular hydrogen produced from a renewable resource.

### BRIEF SUMMARY

A process for producing toluene is provided. The process comprises contacting a hydrocarbonaceous feed stream with a dehydrogenation catalyst to produce a dehydrogenated effluent stream. The dehydrogenated effluent stream is separated into a vapor stream comprising hydrogen and a liquid stream. The liquid stream is fractionated to provide a fractionator overhead stream comprising C7-hydrocarbons and a fractionator bottoms stream comprising toluene. The fractionator overhead stream is passed to a stabilizer column to provide an offgas stream.

### BRIEF DESCRIPTION OF THE DRAWINGS

The FIGURE is a schematic drawing of an exemplary embodiment of a process and apparatus of the present disclosure.

### DEFINITIONS

The term “communication” means that fluid flow is operatively permitted between enumerated components, which may be characterized as “fluid communication”.

The term “downstream communication” means that at least a portion of fluid flowing to the subject in downstream communication may operatively flow from the object with which it fluidly communicates.

The term “upstream communication” means that at least a portion of the fluid flowing from the subject in upstream communication may operatively flow to the object with which it fluidly communicates.

The term “direct communication” or “directly” means that fluid flow from the upstream component enters the downstream component without passing through any other intervening vessel.

The term “indirect communication” means that fluid flow from the upstream component enters the downstream component after passing through an intervening vessel.

The term “column” means a distillation column or columns for separating one or more components of different volatilities. Unless otherwise indicated, each column includes a condenser on an overhead of the column to condense and reflux a portion of an overhead stream back to the top of the column and a reboiler at a bottom of the column to vaporize and send a portion of a bottoms stream back to the bottom of the column. Feeds to the columns may be preheated. The top pressure is the pressure of the overhead vapor at the vapor outlet of the column. The bottom temperature is the liquid bottom outlet temperature. Overhead lines and bottoms lines refer to the net lines from the column downstream of any reflux or reboil to the column. Stripper columns may omit a reboiler at a bottom of the column and instead provide heating requirements and separation impetus from a fluidized inert media such as steam. Stripping columns typically feed a top tray and take main product from the bottom.

As used herein, the term “a component-rich stream” means that the rich stream coming out of a vessel has a greater concentration of the component than the feed to the vessel.

As used herein, the term “a component-lean stream” means that the lean stream coming out of a vessel has a smaller concentration of the component than the feed to the vessel.

As used herein, the term “separator” means a vessel which has an inlet and at least an overhead vapor outlet and a bottoms liquid outlet and may also have an aqueous stream outlet from a boot. A flash drum is a type of separator which may be in downstream communication with a separator that may be operated at higher pressure.

The term “Cx” is to be understood to refer to molecules having the number of carbon atoms represented the subscript “x”. Similarly, the term “Cx−” refers to molecules that contain less than or equal to x and preferably x and less carbon atoms. The term “Cx+” refers to molecules with more than or equal to x and preferably x and more carbon atoms.

### DETAILED DESCRIPTION

The liquid organic hydrogen carrier (LOHC) process involves hydrogenating toluene to methylcyclohexane (MCH) at a first location, transferring the MCH to a second location, and dehydrogenating the MCH to toluene and hydrogen at the second location. To ensure minimal loss of hydrogen, the dehydrogenation process must be performed with no by-products or efficiently removing the by-products. MCH acts as a liquid organic hydrogen carrier, and it can be transferred in storage vessels and/or pipelines for several thousands of miles to the final destination with very minimal to no degradation. The LOHC process helps address the supply and demand gap of blue and green hydrogen as well as the huge differential cost of production between regions.

The present disclosure provides a process and apparatus to optimize the purification loop/system for separating hydrogen from MCH to minimize the energy consumption, maximize the hydrogen and toluene yield and the sustainability of the process.

A hydrocarbon stream comprising methylcyclohexane may be produced at a location and transferred to a second location. The methylcyclohexane feed stream from the storage tanks which may not be completely dry or nitrogen-blanketed may be treated in an oxygen stripper before being routed to the dehydrogenation reactor section. The methylcyclohexane feed may be mixed with a recycle methylcyclohexane stream for example which may be taken from a purification section. The methylcyclohexane feed may be mixed with the hydrogen and preheated for example by exchange with a reactor effluent stream. The temperature of the methylcyclohexane feed may be raised to the reaction temperature in the convection section of a charge heater and passed to the dehydrogenation reactor section. The dehydrogenation reactor section may comprise one or more dehydrogenation reactor(s). The methylcyclohexane feed is charged to the dehydrogenation reactor(s). The dehydrogenation reactor(s) may be radial flow reactors. Interheaters may be used to raise the reactor effluent back to the desired reactor inlet temperature for the next dehydrogenation reactor. The effluent from the last dehydrogenation reactor is cooled in a combined feed exchanger and the product condenser before passing to the separator.

In the separator, the reactor effluent is separated into vapor and liquid streams. The separator vapor may be separated into net gas and recycle gas. The recycle gas may be sent back to be mixed with the feed. The net gas is the hydrogen gas product stream which may be passed to the hydrogen gas compression section. Toluene-rich liquid from the separator is pumped to the purification section.

The hydrogen gas compression section may comprise one or more hydrogen gas compressor(s) which provide sufficient pressure to meet hydrogen purity requirements. The hydrogen purity increases through each stage of compression.

The purification section may comprise a deheptanizer column and a stabilizer column. The present disclosure provides passing the toluene-rich liquid from the separator first to the deheptanizer column. Applicants found that placing the deheptanizer column upstream of the stabilizer significantly reduces the reflux stream to both the deheptanizer column and the stabilizer column. Also, the disclosed process reduces the number of stages required in the deheptanizer column for the separation of toluene. Also, the deheptanizer column is operated at a pressure as low as possible order to minimize reboiler duty and the column temperature. Moreover, the purification section of the present disclosure comprises a rerun column to remove heavy contaminants. A vent compressor is required to recover the small vapor stream from the deheptanizer column and deliver it to the fuel gas system. The rerun column may also be operated at a pressure as low as possible to be able to recover as much toluene as possible.

In the deheptanizer column, toluene is separated in the toluene-rich liquid bottoms stream. The overhead stream comprising C7-hydrocarbons and light gases is condensed and passed to the stabilizer column. A reflux stream is taken from the condensed liquid stream. The stabilizer column separates offgas comprising hydrogen, methane and traces of C5-6 hydrocarbons from a liquid stream comprising C6-7 hydrocarbons, dimethylcyclopentane (DMCP), and ethylcyclopentane (ECP). The offgas stream can be sent to the refinery or used as fuel gas, for example.

The toluene-rich liquid bottoms stream may be passed to the rerun column to separate a heavy stream comprising C8-16 hydrocarbons and provide a toluene product stream.

FIGURE illustrates an exemplary embodiment of the process of producing toluene 100. The process 100 comprises a dehydrogenation reaction section 101 and a purification section 111. A hydrocarbonaceous stream is taken from a storage tank in line 102 and passed to the dehydrogenation reaction section 101. In an aspect, the hydrocarbonaceous stream in line 102 may be taken from a saturation unit. The feed stream to the saturation unit may comprise an aromatic hydrocarbons stream and a hydrogen stream produced from renewable sources for example “green hydrogen” or “blue hydrogen”. In an exemplary embodiment, the hydrocarbonaceous stream in line 102 comprises MCH. In another exemplary embodiment, the hydrocarbonaceous stream in line 102 comprises one or both of MCH and toluene. In an aspect, the dehydrogenation reaction section 101 may comprise an oxygen stripper 110, a combined feed exchanger 115, a charge heater 117, and a dehydrogenation reactor 120.

The hydrocarbonaceous stream in line 102 is passed to the oxygen stripper 110 to remove oxygen and provide a stripped hydrocarbonaceous stream in line 112. The stripped hydrocarbonaceous stream in line 112 may be combined with a recycle hydrogen stream in line 133 forming a combined feed stream in line 114. The combined feed stream in line 114 may be passed to a combined feed exchanger 115 to preheat the combined feed stream in line 114 by heat exchange with a dehydrogenation reactor effluent stream in line 124. In an embodiment, the oxygen stripper 110 is optional and the hydrocarbonaceous stream in line 102 may be combined with the recycle hydrogen stream to provide the combined feed stream in line 114. A preheated combined feed stream is taken in line 116 from the combined feed exchanger 115. The preheated combined feed stream in line 116 may be further heated in a charge heater 117 to the reactor temperature. A heated combined feed stream is taken in line 118 from the charge heater 117 and passed to the dehydrogenation reactor 120. The dehydrogenation reactor 120 may comprise one or more dehydrogenation catalyst bed(s). In an aspect, the dehydrogenation reactor 120 may comprise one or more dehydrogenation reactor vessels to dehydrogenate the heated combined feed stream. In another aspect, the dehydrogenation reaction section 101 may comprise one or more interheater(s) 122 to heat an intermediate reactor effluent stream in line 121 to the desired reactor inlet temperature between dehydrogenation reactors. A heated intermediate reactor effluent stream may be taken in line 123 from the interheater 122 and sent back to the dehydrogenation reactor 120.

The hydrocarbonaceous feed 102 typically comprises methylcyclohexane (MCH). This stream may be produced from a number of different processes. In the exemplary embodiment, it is envisioned that the MCH may be produced in a toluene saturation unit that will chemically bind green or blue hydrogen to a toluene stream. It is further envisioned that this toluene stream may be chemically similar to a toluene product stream of the process as described later in detail or be the toluene product stream of the process itself.

Any suitable dehydrogenation catalyst that can achieve a selectivity in the dehydrogenation of methylcyclohexane to toluene and hydrogen in excess of 99.8% can be used in the dehydrogenation reactor 120. Suitable dehydrogenation catalysts may include, but are not limited to, alumina, a noble metal, and an alkali or alkaline earth metal. Suitable noble metals include, but are not limited to, platinum, palladium, rhodium, ruthenium, rhenium, iridium, gold, osmium, silver, or combinations thereof. Suitable alkali or alkaline earth metals include, but are not limited to, sodium, cesium, potassium, rubidium, francium, lithium, beryllium, strontium, barium, calcium, magnesium, radium, or combinations thereof.

A dehydrogenated reactor effluent stream is taken in line 124 from the dehydrogenation reactor 120 and passed to the combined feed exchanger 115. A heat exchanged perhaps cooled dehydrogenated reactor effluent stream is taken in line 126 from the combined feed exchanger 115. The heat exchanged dehydrogenated reactor effluent stream in line 126 may be passed to a cooler 127 to provide a further cooled dehydrogenated reactor effluent stream in line 128. The reactions taking place in the dehydrogenation reactor 120 may include a main reaction of dehydrogenation of the MCH to toluene releasing hydrogen and may include one or more side reactions. One side reaction includes cracking of toluene to benzene and methane. Another side reaction includes isomerization of methylcyclohexane to dimethylcyclopentane (DMCP), and ethylcyclopentane (ECP). A third side reaction includes the dimerization of methylcyclohexanes to heavier molecules, such as dimethyl diphenyl compounds. These side reactions affect the overall yield and the purity of the products streams of the process 100. The purification section 111 separates byproducts from the products streams to promote the purity of the product streams while consuming a minimum energy in the separation.

The dehydrogenation reaction section 101 further comprises a separator 130 to separate the reactor effluent into vapor and liquid streams. The cooled dehydrogenated reactor effluent stream in line 126 is passed to the separator 130 where it is separated into a separator vapor stream comprising hydrogen in line 132 and a separator liquid stream comprising toluene in line 134. The recycle hydrogen stream is taken in line 133 from the separator vapor stream in line 132. The remaining portion of the separator vapor stream is taken in line 137 and passed to a hydrogen compressor 135 where it is compressed to provide a hydrogen product stream which is taken in line 136.

The separator liquid stream in line 134 is a toluene rich liquid stream, and toluene is separated from this stream. The separator liquid stream in line 134 is passed to the purification section 111.

In an embodiment, the purification section 111 comprises a fractionation column 140, a stabilizer column 160, and a rerun column 170.

In an embodiment, separator liquid stream in line 134 is fractionated in the fractionation column 140 to provide a fractionator overhead stream in line 142 and a fractionator bottoms stream in line 143. In an exemplary embodiment, the fractionation column 140 is a deheptanizer column. The fractionator overhead stream in line 142 may comprise C7-hydrocarbons. In an embodiment, the fractionator overhead stream in line 142 may comprise benzene and C7-saturated hydrocarbons including hexanes, methylcyclopentane, cyclohexane, heptane, dimethylcyclopentane, methylcyclohexane, and ethylcyclopentane. In another embodiment, the fractionator overhead stream in line 142 may comprise light hydrocarbons, for example C3-hydrocarbons, and/or C2-hydrocarbons. Some toluene may slip to the fractionator overhead stream in line 142. The fractionator overhead stream in line 142 may also comprise trace amounts of hydrogen and methane.

In an exemplary embodiment, the fractionation column 140 may be operated at an overhead pressure of about 34 kPa (g) (5 psig) to about 207 kPa (g) (30 psig) and a bottoms temperature of about 93° C. (200° F.) to about 177° C. (350° F.). In an aspect, the heat to the fractionation column 140 may be supplied from a reboiler 141 using steam. Alternatively, a hot oil system or any suitable hot fluid medium may be used for the reboiler 141. Operating the fractionation column 140 at a lower pressure minimizes the duty of the reboiler 141.

The fractionator overhead stream in line 142 may be passed to the stabilizer column. In an aspect, the fractionator overhead stream in line 142 may be combined with a stabilizer column overhead stream in line 162 to provide a combined overhead stream in line 146. The combined overhead stream in line 146 may be cooled in an overhead heat exchanger 147 and a cooled overhead stream in line 148 is passed to an overhead receiver 150. The cooled overhead stream in line 148 may be totally or partially condensed in the overhead receiver 150 to provide a condensed overhead liquid stream in line 155 and an offgas stream in line 152. A reflux stream is taken in line 157 from the condensed overhead liquid stream in line 155. In an embodiment, the reflux stream in line 157 may be passed to the fractionation column 140 at a reflux to feed ratio of about 0.02 to 0.5. In another embodiment, the reflux stream in line 157 may be passed to the fractionation column 140 at a reflux to feed ratio of about 0.03 to 0.4. In another embodiment, the reflux stream in line 157 may be passed to the fractionation column 140 at a reflux to feed ratio of about 0.04 to 0.3 or about 0.05 to 0.2. In yet another embodiment, the reflux stream in line 157 may be passed to the fractionation column 140 at a reflux to feed ratio of about 0.1 to about 0.1.

For complete conversion of the methylcyclohexane to toluene and hydrogen in the dehydrogenation reactor 120, the separator liquid stream in line 134 may not comprise a significant amount of methylcyclohexane. The present process does not require a recycle stream of methylcyclohexane from the fractionation column 140 to the dehydrogenation reactor 120 for complete conversion in the dehydrogenation reactor 120. With no recycle stream from the fractionation column 140, the fractionation column of the current process may be run at a much lower reflux to feed ratio. Also, the fractionation column 140 of the current process requires a much smaller number of stages even as low as 50% of the number of stages when compared with a fractionation column with a recycle stream. For the situation with limited methylcyclohexane conversion in the dehydrogenation reactor such as revamps, some methylcyclohexane may be present in the separator liquid stream in line 134. Under limited conversion conditions, a recycle stream may be taken from the fractionation column and passed to the dehydrogenation reactor 120.

A fractionator overhead liquid stream is taken in line 156 from the condensed overhead liquid stream in line 155. The fractionator overhead liquid stream in line 156 is a net liquid stream from the overhead receiver 150. The net liquid stream in line 156 is passed to the stabilizer column 160. The stabilizer column removes the byproducts and/or impurities from the process in a bottoms stream in line 164. In an aspect, the stabilizer column bottoms stream in line 164 may comprise benzene and C7-saturated hydrocarbons such as hexane, methylcyclopentane, cyclohexane, heptane, dimethylcyclopentane, methylcyclohexane, and ethylcyclopentane. The stabilizer column overhead stream in line 162 may comprise hydrogen and methane. The stabilizer column overhead stream in line 162 may be recycled. The stabilizer column overhead stream in line 162 may be combined with the fractionator overhead stream in line 142 to provide the combined overhead stream in line 146 and passed to the overhead receiver 150. The offgas stream may be taken in line 152 from the overhead receiver 150, compressed in an overhead compressor 153 to provide a compressed offgas stream in line 154. The compressed offgas stream in line 154 may comprise hydrogen and methane. The compressed offgas stream in line 154 may be used in the process, for example, burned to provide heat to the reactor or the column or it may be sent to an off-site utility. In an aspect, at least a portion of the compressed offgas stream in line 154 may be recycled to the hydrogen compressor 135. The compressed offgas stream in line 154 may be recycled to the hydrogen compressor 135 with the separator vapor stream in line 137 to recover hydrogen into the hydrogen product stream 136. In an embodiment, about 50 vol % to about 70 vol % of the compressed offgas stream in line 154 may be recycled to the hydrogen compressor 135.

In an embodiment, the stabilizer column 160 may be operated at an overhead pressure of about 34 kPa (g) (5 psig) to about 207 kPa (g) (30 psig) and a bottoms temperature of about 65° C. (150° F.) to about 122° C. (250° F.). In an aspect, the heat to the stabilizer column 160 may be supplied from a reboiler 161 using steam. Alternatively, a hot oil system or any suitable hot fluid medium may be used for the reboiler 161.

Referring back to the fractionation column 140, the fractionator bottoms stream in line 143 may be separated to provide a first liquid stream in line 144 and a second liquid stream in line 145. The fractionator bottoms stream in line 143 may comprise aromatic hydrocarbons. Preferably, the fractionator bottoms stream in line 143 may comprise toluene. The first liquid stream in line 144 may be further processed to separate the toluene. In an embodiment, the first liquid stream in line 144 may be passed to the rerun column 170 to separate heavies from the toluene. In an embodiment, the rerun column 170 may be operated at an overhead pressure of below atmospheric pressure or vacuum pressure. In an exemplary embodiment, the rerun column 170 may be operated at an overhead pressure of about −69 kPa (g) (−10 psig) to about 95 kPa(a) (13.8 psia) or about −6 kPa (g) (−1 psig) to about 32 kPa(a) (4.7 psia). In another exemplary embodiment, the rerun column 170 may be operated at atmospheric pressure. In an embodiment, the rerun column 170 may be operated at a bottom temperature of about 65° C. (150° F.) to about 149° C. (300° F.). In an aspect, the heat to the rerun column 170 may be supplied from a reboiler 171 using steam. Alternatively, a hot oil system or any suitable hot fluid medium may be used for the reboiler 171. A heavy stream is separated in the rerun column 170 bottoms and taken in line 174. In an embodiment, the heavy stream in line 174 may comprise C8-C16 aromatics and multi-ring aromatics but may also include some toluene.

A rerun overhead stream comprising toluene is taken in line 172 from the rerun column 170. The rerun overhead stream comprising toluene in line 172 may be cooled in a heat exchanger 173 and a cooled rerun overhead stream is passed to a rerun overhead receiver 175. The rerun overhead stream 172 is further cooled in the rerun overhead receiver 175 to provide a toluene stream in line 176. A vacuum pump 178 is provided at the overhead of the rerun column 170 on the stream 177 from the overhead receiver 175 to operate the rerun column 170 at a pressure as low as possible. The recovered toluene stream in line 176 may be combined with the second liquid stream in line 145 to provide a toluene product stream in line 149.

While the foregoing embodiment envisages the use of methylcyclohexane (MCH) and toluene as the liquid organic hydrogen carrier (LOHC), the alternative, similar LOHC systems may benefit from the disclosed process. Examples may include cyclohexane and benzene; dimethylcyclohexanes and xylenes; and trimethylcyclohexanes and trimethylbenzenes.

The foregoing process and apparatus provide an efficient way of producing hydrogen from a hydrocarbonaceous stream.

### EXAMPLE

A simulation study was performed to compare an exemplary embodiment of the process of the present disclosure with another process wherein a recycle stream was taken from the deheptanizer column 140 to the dehydrogenation reactor 120. For this study, the feed stream to both the process had the same concentration of heavies, ethylcyclopentane, and dimethylcyclopentane. The results including the various parameters, yield, and heat duty of the column are summarized in Table 1 below:

TABLE 1

| | Exemplary embodiment of the | | | | | |
|---|---|---|---|---|---|---|
| | present disclosure | | | Process with recycle stream | | |
| | Deheptanizer | Stabilizer | Rerun | Deheptanizer | Stabilizer | Rerun |
| No. of stages | 32 | 5 | 10 | 60 | 5 | 10 |
| Column overhead | 104 | 83 | −46 | 104 | 83 | -46 |
| pressure, kPa(g) | | | | | | |
| Reflux/feed ratio | 0.15 | — | — | 1.85 | — | — |
| Reboiler duty, | 13.3 | 0.05 | 10.5 | 55.7 | 0.05 | 5.9 |
| MMBTU/hr | | | | | | |
| Condenser duty, | 5.2 | — | 15.35 | 52.3 | | 5.95 |
| MMBTU/hr | | | | | | |
| Feed-bottoms | 6.9 | — | — | 10.95 | — | — |
| exchanger duty | | | | | | |

% Recovery from Toluene purification section

| | | |
|---|---|---|
| Toluene recovery | 99.9 | 98.2 |
| C7 cyclopentanes | 20.5 | 20 |
| rejection | | |
| Heavies rejection | 49.8 | 19.7 |

The results in Table 1 show that the current process required much lower deheptanizer column reboiler duty. Also, the current process provided a higher toluene recovery and higher (over 200%) heavies rejection from the process.

### SPECIFIC EMBODIMENTS

While the following is described in conjunction with specific embodiments, it will be understood that this description is intended to illustrate and not limit the scope of the preceding description and the appended claims.

A first embodiment of the present disclosure is a process of producing aromatic hydrocarbons, comprising contacting a hydrocarbonaceous feed stream with a dehydrogenation catalyst to produce a dehydrogenated effluent stream; separating the dehydrogenated effluent stream into a vapor stream comprising hydrogen and a liquid stream; fractionating the liquid stream to provide a fractionator overhead stream and a fractionator bottoms stream; and passing the fractionator overhead stream to a stabilizer column to provide an offgas stream. An embodiment of the present disclosure is one, any or all of prior embodiments in this paragraph up through the first embodiment in this paragraph, wherein the hydrocarbonaceous feed stream comprises methylcyclohexane. An embodiment of the present disclosure is one, any or all of prior embodiments in this paragraph up through the first embodiment in this paragraph, wherein the hydrocarbonaceous feed stream comprises aromatic hydrocarbons. An embodiment of the present disclosure is one, any or all of prior embodiments in this paragraph up through the first embodiment in this paragraph wherein the stabilizer column also provides a bottoms stream comprising C7 cyclopentanes. An embodiment of the present disclosure is one, any or all of prior embodiments in this paragraph up through the first embodiment in this paragraph further comprising separating a heavy stream from the fractionator bottoms stream. 5 An embodiment of the present disclosure is one, any or all of prior embodiments in this paragraph up through the first embodiment in this paragraph further comprising separating the fractionator bottoms stream into a first liquid stream and a second liquid stream; fractionating the first liquid stream in a rerun column to separate heavies from a recovered aromatics; and combining the recovered aromatics with the second liquid stream to provide an aromatics product stream. An embodiment of the present disclosure is one, any or all of prior embodiments in this paragraph up through the first embodiment in this paragraph, wherein the first liquid stream is fractionated in a rerun column operating at an overhead pressure of about 32 kPa(a) (4.7 psia) to about 95 kPa(a) (13.8 psia). An embodiment of the present disclosure is one, any or all of prior embodiments in this paragraph up through the first embodiment in this paragraph further comprising fractionating the first liquid stream into an overhead stream comprising toluene and a bottoms stream heavy stream; condensing the overhead stream comprising toluene to provide a toluene stream and a bottoms gas stream; and combining the toluene stream with the second liquid stream to provide a toluene product stream. An embodiment of the present disclosure is one, any or all of prior embodiments in this paragraph up through the first embodiment in this paragraph further comprising combining a fractionated overhead stream with a stabilizer column overhead stream to provide a combined overhead stream; condensing the combined overhead stream to provide a condensed overhead liquid and the offgas stream; and passing a reflux stream taken from the condensed overhead liquid to the fractionation column. An embodiment of the present disclosure is one, any or all of prior embodiments in this paragraph up through the first embodiment in this paragraph wherein the reflux stream is passed to the fractionation column at a reflux to feed ratio of about 0.05 to about 0.5. An embodiment of the present disclosure is one, any or all of prior embodiments in this paragraph up through the first embodiment in this paragraph, further comprising taking a net liquid stream from the condensed overhead liquid as the fractionator overhead stream. An embodiment of the present disclosure is one, any or all of prior embodiments in this paragraph up through the first embodiment in this paragraph, wherein the rerun column is operated at a vacuum pressure. An embodiment of the present disclosure is one, any or all of prior embodiments in this paragraph up through the first embodiment in this paragraph, wherein the dehydrogenation catalyst comprises Al₂O3, a noble metal, and an alkali or alkaline earth metal. An embodiment of the present disclosure is one, any or all of prior embodiments in this paragraph up through the first embodiment in this paragraph further comprising separating the vapor stream into a recycle hydrogen stream and a product hydrogen stream for further compression and distribution. An embodiment of the present disclosure is one, any or all of prior embodiments in this paragraph up through the first embodiment in this paragraph, wherein the hydrocarbonaceous feed stream is taken from a saturation unit, and wherein a feed stream to the saturation unit comprises an aromatic hydrocarbons stream and a hydrogen stream produced from renewable sources.

A second embodiment of the present disclosure is a process of producing aromatic hydrocarbons, comprising contacting a hydrocarbonaceous feed stream with a dehydrogenation catalyst to produce a dehydrogenated effluent stream; separating the dehydrogenated effluent stream into a vapor stream comprising hydrogen and a liquid stream; fractionating the liquid stream in a fractionation column to provide a fractionator overhead stream and a fractionator bottoms stream comprising aromatics hydrocarbons; and passing the fractionator overhead stream to a stabilizer column to provide an offgas stream. An embodiment of the present disclosure is one, any or all of prior embodiments in this paragraph up through the second embodiment in this paragraph, wherein the fractionator bottoms stream comprises toluene. An embodiment of the present disclosure is one, any or all of prior embodiments in this paragraph up through the second embodiment in this paragraph, wherein the hydrocarbonaceous feed stream comprises methylcyclohexane.

A third embodiment of the present disclosure is a process of producing aromatic hydrocarbons, comprising contacting a hydrocarbonaceous feed stream comprising methylcyclohexane with a dehydrogenation catalyst to produce a dehydrogenated effluent stream; separating the dehydrogenated effluent stream into a vapor stream comprising hydrogen and a liquid stream; fractionating the liquid stream in a fractionation column to provide a fractionator overhead stream and a fractionator bottoms stream comprising aromatics hydrocarbons; and passing the fractionator overhead stream to a stabilizer column to provide an offgas stream. An embodiment of the present disclosure is one, any or all of prior embodiments in this paragraph up through the third embodiment in this paragraph, wherein the fractionator bottoms stream comprises toluene.

Without further elaboration, it is believed that using the preceding description that one skilled in the art can utilize the present disclosure to its fullest extent and easily ascertain the essential characteristics of this disclosure, without departing from the spirit and scope thereof, to make various changes and modifications of the present disclosure and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

1. A process of producing aromatic hydrocarbons, comprising:
contacting a hydrocarbonaceous feed stream with a dehydrogenation catalyst to produce a dehydrogenated effluent stream;
separating said dehydrogenated effluent stream into a vapor stream comprising hydrogen and a liquid stream;
fractionating said liquid stream in a fractionation column to provide a fractionator overhead stream and a fractionator bottoms stream; and
passing said fractionator overhead stream to a stabilizer column to provide an offgas stream.

2. The process of claim 1, wherein said hydrocarbonaceous feed stream comprises methylcyclohexane.

3. The process of claim 1, wherein said hydrocarbonaceous feed stream comprises aromatic hydrocarbons.

4. The process of claim 1 wherein the stabilizer column provides a bottoms stream comprising C7-hydrocarbons.

5. The process of claim 1 further comprising separating a heavy stream from said fractionator bottoms stream.

6. The process of claim 5 further comprising:
separating said fractionator bottoms stream into a first liquid stream and a second liquid stream;
fractionating said first liquid stream in a rerun column to separate heavies from a recovered aromatics stream; and
combining said recovered aromatics stream with said second liquid stream to provide an aromatic product stream.

7. The process of claim 6, wherein said first liquid stream is fractionated in a rerun column operating at an overhead gauge pressure of about 32 kPa(a) (4.7 psia) to about 95 kPa(a) (13.8 psia).

8. The process of claim 6 further comprising:
fractionating said first liquid stream into an overhead stream comprising toluene and a bottoms stream heavy stream;
condensing said overhead stream comprising toluene to provide a toluene stream and a bottoms gas stream; and
combining said toluene stream with said second liquid stream to provide a toluene product stream.

9. The process of claim 1 further comprising:
combining a fractionated overhead stream with a stabilizer column overhead stream to provide a combined overhead stream;
condensing said combined overhead stream to provide a condensed overhead liquid stream and said offgas stream; and
passing a reflux stream taken from said condensed overhead liquid stream to the fractionation column.

10. The process of claim 9, wherein the reflux stream is passed to the fractionation column at a reflux to feed ratio of about 0.05 to about 0.5.

11. The process of claim 9, further comprising taking a net liquid stream from said condensed overhead liquid as said fractionator overhead stream.

12. The process of claim 7, wherein the rerun column is operated at a vacuum pressure.

13. The process of claim 1, wherein the dehydrogenation catalyst comprises alumina, a noble metal, and an alkali or alkaline earth metal.

14. The process of claim 1 further comprising separating said vapor stream into a recycle hydrogen stream and a product hydrogen stream for further compression and distribution.

15. The process of claim 1 wherein said hydrocarbonaceous feed stream is taken from a saturation unit, and wherein a feed stream to the saturation unit comprises an aromatic hydrocarbons stream and a hydrogen stream produced from renewable sources.

16. A process of producing aromatic hydrocarbons, comprising:
contacting a hydrocarbonaceous feed stream with a dehydrogenation catalyst to produce a dehydrogenated effluent stream;
separating said dehydrogenated effluent stream into a vapor stream comprising hydrogen and a liquid stream;
fractionating said liquid stream in a fractionation column to provide a fractionator overhead stream and a fractionator bottoms stream comprising aromatics hydrocarbons; and
passing said fractionator overhead stream to a stabilizer column to provide an offgas stream.

17. The process of claim 16, wherein said fractionator bottoms stream comprises toluene.

18. The process of claim 16, wherein said hydrocarbonaceous feed stream comprises methylcyclohexane.

19. A process of producing aromatic hydrocarbons, comprising:
contacting a hydrocarbonaceous feed stream comprising methylcyclohexane with a dehydrogenation catalyst to produce a dehydrogenated effluent stream;
separating said dehydrogenated effluent stream into a vapor stream comprising hydrogen and a liquid stream;
fractionating said liquid stream in a fractionation column to provide a fractionator overhead stream and a fractionator bottoms stream comprising aromatics hydrocarbons; and
passing said fractionator overhead stream to a stabilizer column to provide an offgas stream.

20. The process of claim 19, wherein said fractionator bottoms stream comprises toluene.
